(19) United States
(12) Patent Application Publication
(10) Pub. No.: US 2017/0196594 A1
(43) Pub. Date: Jul. 13, 2017

(54) DEVICE FOR ASSISTING DELIVERY OF AN INFANT DURING CHILDBIRTH

(71) Applicant: BECTON DICKINSON HOLDINGS PTE LTD, Singapore (SG)

(72) Inventors: Tirza SUMITRO, Singapore (SG); Darius ALISANTOSO, Singapore (SG); Chong Yong KOH, Singapore (SG)

(21) Appl. No.: 15/325,026

(22) PCT Filed: Jul. 29, 2014
(86) PCT No.: PCT/SG2014/000359

§ 371 Date: Jan. 9, 2017

(51) Int. Cl. A61B 17/44

(52) U.S. Cl. CPC A61B 17/442; A61B 2017/00287

(57) ABSTRACT

A device for assisting delivery of an infant during childbirth includes a sleeve configured to be positioned over and around the infant's head, an inserter comprising a handle provided at a proximal end of the inserter, flexible arms attached to the handle and configured to engage and position the sleeve over and around the infant's head, a central rod attached to the handle, an inner tube provided around the central rod at a distal end of the inserter such that the inner tube is retracted over the central rod when a force is applied to a soft cone provided at a distal end of the inner tube, the soft cone being configured to be placed on the infant's head to position the device for inserting the sleeve over and around the infant's head, and a reuse prevention feature configured to prevent the inner tube from being retracted a second time.

### FIELD OF THE INVENTION

This invention relates to a device for assisting delivery of an infant during childbirth.

### BACKGROUND OF THE INVENTION

PCT publication number WO 2012/164139 describes a device to extract an element contained in a cavity. The device may be used for assisting delivery of an infant during childbirth when the infant is in a cephalic or head-first presentation. The device comprises a bag or sleeve that is a tube of flexible material folded onto itself. The device also comprises an applier or inserter configured to insert the sleeve through the birth canal and to position the sleeve over and around the infant's head. After the infant has been delivered, the device may potentially be washed and reused by deflating the air chamber and resetting the inserter and sleeve for another use. However, in some cases, it may be desirable to prevent reuse of the device.

In addition, although the device described in PCT publication number WO 2012/164139 provides an openwork window on the inserter through which a scale or other marker may be viewed to display how deeply the sleeve has entered to surround the infant's head, some users may need additional feedback as to when pushing on the inserter should cease once the sleeve has been correctly positioned.

### SUMMARY OF INVENTION

According to an exemplary aspect, there is provided a device for assisting delivery of an infant during childbirth, the device comprising a sleeve configured to be positioned over and around the infant's head for exerting traction on the infant therewith; and an inserter comprising a handle provided at a proximal end of the inserter, flexible arms attached to the handle and configured to engage and position the sleeve over and around the infant's head, a central rod attached to the handle, an inner tube provided around the central rod at a distal end of the inserter such that the inner tube is retracted over the central rod when a force is applied to a soft cone provided at a distal end of the inner tube, the soft cone being configured to be placed on the infant's head to position the device for inserting the sleeve over and around the infant's head, and a reuse prevention feature configured to prevent the inner tube from being retracted a second time after a single use of the device.

The reuse prevention feature may comprise a moveable collar provided at least partially within the distal end of the handle around the central rod, the moveable collar configured to be moved by the inner tube, and an elastic projection attached to the handle, the elastic projection extending distally and configured to engage the central rod, wherein, before use of the device, the moveable collar engages the elastic projection, and after a single use of the device, the moveable collar is moved by the inner tube away from the elastic projection such that the elastic projection engages the central rod when the inner tube is returned to an unretracted position to prevent a second retraction of the inner tube.

The elastic projection may comprise a number of flat springs projecting from a distal end of a stationary collar attached to a distal end of the handle around the moveable collar, the distal end of each flat spring being biased inwardly towards the central rod.

The device may further comprise an opening provided in the handle and a button attached to and biased outwardly from a moveable part provided in the handle, the moveable part configured to be moved by the inner tube when the inner tube is retracted during use of the device such that the button is aligned with and pops up in the opening when the inner tube is fully retracted.

The moveable part may be the moveable collar.

The device may further comprise a button biaser configured to attach and bias the button outwardly away from the moveable part.

### BRIEF DESCRIPTION OF FIGURES

In order that the invention may be fully understood and readily put into practical effect there shall now be described by way of non-limitative example only exemplary embodiments of the present invention, the description being with reference to the accompanying illustrative drawings.

FIG. 1 is a perspective view of a first exemplary embodiment of a device of the present invention;

FIG. 2 is the device of FIG. 1 with a flexible arm hidden and a handle shown in section view to show inner components of the device;

FIG. 3a is a sectional side view of the device of FIG. 1 with an inner tube in an unretracted initial position;

FIG. 3b is a close-up sectional side view of a reuse prevention feature of the device in FIG. 3a;

FIG. 4a is a sectional side view of the device of FIG. 1 with the inner tube in a partially retracted position;

FIG. 4b is a close-up sectional side view of the reuse prevention feature of the device in FIG. 4a;

FIG. 4c is a close up sectional side view of a moveable collar of the reuse prevention feature of FIG. 4b engaging an inner tube of the device;

FIG. 5a is a sectional side view of the device of FIG. 1 with the inner tube in a fully retracted position;

FIG. 5b is a close-up sectional side view of the reuse prevention feature of the device in FIG. 5a;

FIG. 6a is a sectional side view of the device with the inner tube in an unretracted final position;

FIG. 6b is a close-up sectional side view of the reuse prevention feature of the device in FIG. 6a;

FIG. 7 is a sectional side view of a second exemplary embodiment of the device with an inner tube in an unretracted initial position;

FIG. 8a is a sectional side view of the device of FIG. 7 with the inner tube in a partially retracted position;

FIG. 8b is a close-up sectional side view of a button of the device in FIG. 8a;

FIG. 9a is a sectional side view of the device of FIG. 7 with the inner tube in a fully retracted position; and

FIG. 9b is a close-up sectional side view of the button of the device in FIG. 9a.

### DETAILED DESCRIPTION

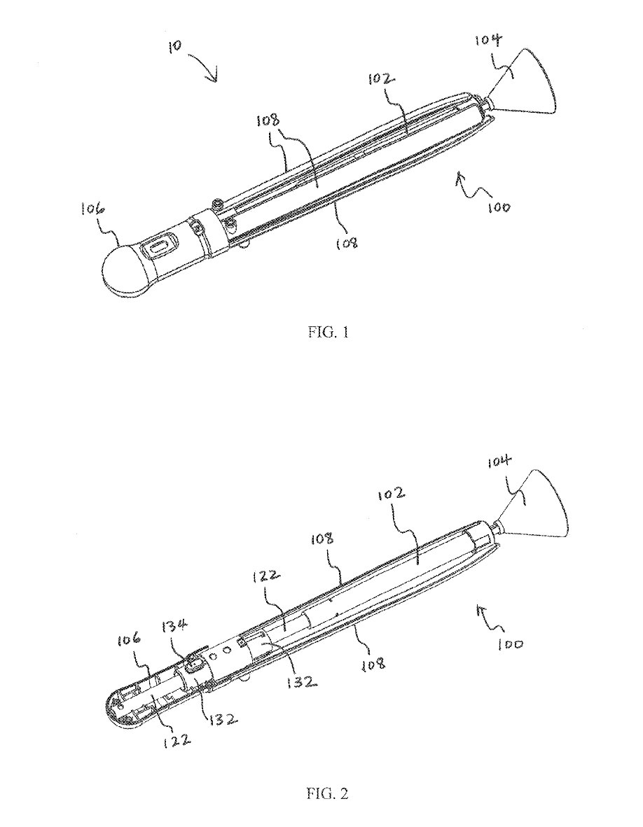

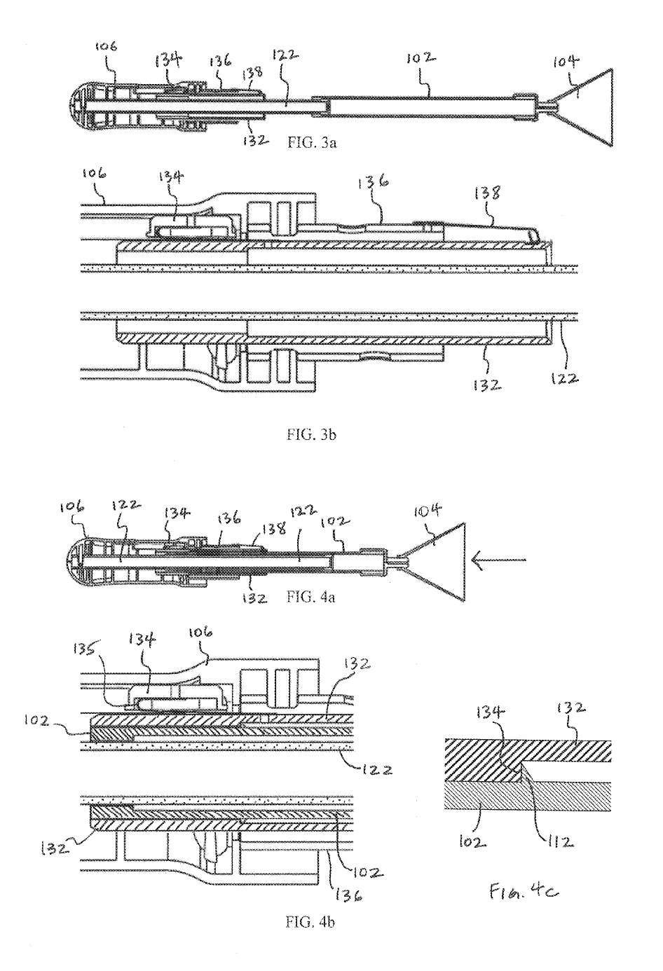

Exemplary embodiments of the invention will be described with reference to FIGS. 1 to 9b. In FIGS. 3a to 9b, flexible arms 108 of the device 10 have been hidden for greater clarity.

In a first embodiment, as shown in FIGS. 1 to 6b, the device 10 comprises a sleeve (not shown) and an inserter 100. The inserter 100 comprises an inner tube 102 provided at a distal end of the inserter 100, the inner tube 102 having a soft cone 104 provided at its distal end. The soft cone 104 is configured to be placed on the infant's head to position the device 10 for inserting the sleeve over and around the infant's head. The inner tube 102 is configured to be retractable relative to a handle 106 provided at a proximal end of the inserter 100. A central rod 122 is connected to the handle 106 and the inner tube 102 is provided around the central rod 122 such that retraction of the inner tube 102 occurs when the inner tube 102 slides over the central rod 122 towards the handle 106 as a result of a force applied to the soft cone 104. A resilient means such as a spring (not shown) is provided to bias the inner tube 102 towards an unretracted position as shown in FIGS. 1, 2, 3a, 3b, 6a, 6b and 7.

The inserter 100 further comprises a number of flexible arms 108. The proximal end of each flexible arm 108 is attached to the handle 106 while the distal end of the each flexible arm 108 is free. The flexible arms 108 are provided around the inner tube 102 and are configured to be inserted, from the distal end, into pockets provided in the sleeve of the device 10 in order to push the sleeve over the soft cone 104 and over and around the infant's head, so that traction may be exerted on the infant via the sleeve.

Before use, the inner tube 102 is at an initial unretracted position outside the handle 106, as shown in FIGS. 1, 2, 3a and 7, and the flexible arms 108 are inserted into the pockets of the sleeve. In this position, the sleeve is around the unretracted inner tube 102 and the soft cone 104 extends distally of the sleeve.

In use, the device 10 is inserted into the birth canal until the soft cone 104 is positioned on the infant's head. By further pushing on the handle 106, the inner tube 102 is retracted towards the handle 106 by being pushed back over the central rod 122 due to the infant's head exerting a force against the soft cone 104 onto the inner tube 102, as shown in FIG. 4a, while the flexible arms 108 and sleeve become relatively extended over the retracted soft cone 104, thereby sliding the sleeve over and around the infant's head.

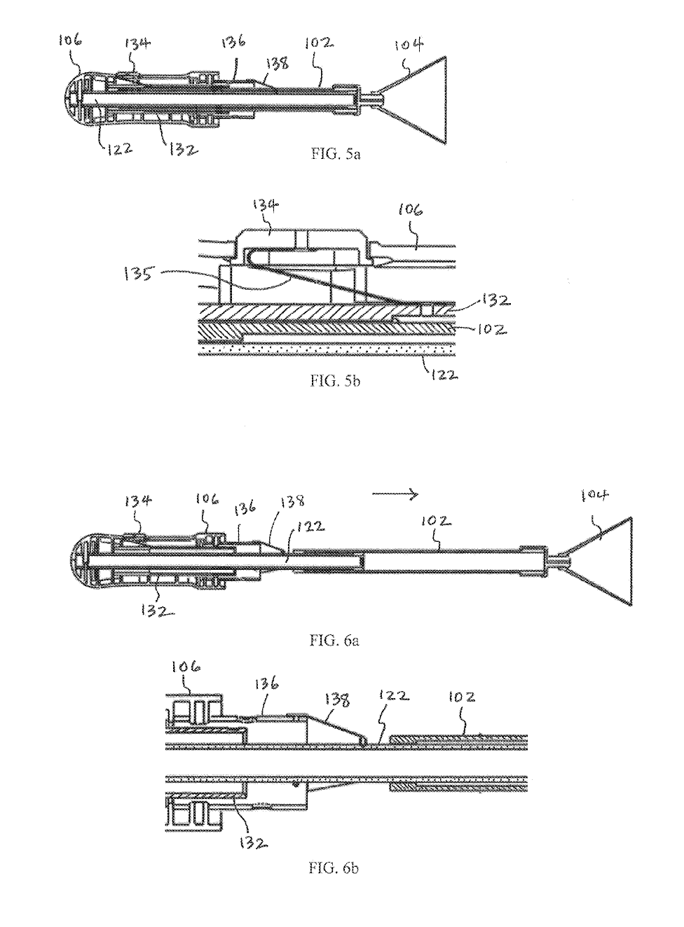

When the sleeve has been inserted to a sufficient depth to be correctly positioned over and around the infant's head, the inner tube 102 is fully retracted as shown in FIGS. 5a, 5b and 8a, 8b.

The inserter 100 is subsequently entirely withdrawn, leaving the sleeve in place for applying traction thereunto in order to assist delivery of the infant. When the inserter 100 has been withdrawn, the soft cone 104 is no longer in contact with the infant's head and the resilient means biases the inner tube 102 back to its final unretracted position as shown in FIG. 6a.

To prevent reuse of the device 10, the inserter 100 is configured such that after a single use, the inner tube 102 cannot be retracted again. In this way, it is not possible for the flexible arms 108 to be relatively extended over the soft cone 104 a second time in order to position the sleeve over another infant's head.

The inner tube 102 is prevented from being retracted a second time after a single use of the device 10 by providing a reuse prevention feature on the inserter 100. In a first exemplary embodiment, as shown in FIGS. 2 to 6b, the reuse prevention feature comprises a moveable collar 132 provided at least partially within the distal end of the handle 106 around the central rod 122, and an elastic projection 138 attached to the handle 106, the elastic projection 138 extending distally and configured to engage the central rod 122. In this embodiment, the elastic projection 138 comprises a number of flat springs 138 that project from a distal end of a stationary collar 136 attached to the handle 106, around the moveable collar 132. The distal end of each flat spring 138 is biased inwardly towards the central rod 122.

The reuse prevention feature is configured such that before use of the device 10, the moveable collar 132 is positioned relative to the stationary collar 136 such that the distal end of the moveable collar 132 extends beyond the distal end of the stationary collar 136. In this position, the moveable collar 132 engages the flat springs 138 in that the distal ends of the flat springs 138 are in contact with the moveable collar 132, as shown in FIGS. 3a and 3b.

During use, the infant's head exerts a force on the soft cone 104, causing the inner tube 102 to retract relative to the handle 106 as indicated by the arrow in FIG. 4a. As the inner tube 102 retracts, it slides over the central rod 122 to contact the moveable collar 132. In the embodiment shown in FIGS. 4a to 4c, the inner tube 102 slides into the moveable collar 132 as it retracts. An inwardly projecting step 134 is provided on an interior surface of the moveable collar 132 to engage a projecting rim 112 provided on an exterior surface of the inner tube 102, as shown in close up in FIG. 4c.

As the inner tube 102 continues to retract, the projecting rim 112 of the inner tube 102 pushes against the projecting step 134 of the moveable collar 132. This action moves the moveable collar 132 into the handle 106 together with retracting inner tube 102, as shown in FIGS. 5a and 5b, in which the inner tube 102 has reached its fully retracted position. In this position, the moveable collar 132 no longer extends past the distal ends of the flat springs 138. Instead, the distal ends of the flat springs 138 are now in contact with the inner tube 102 which has been retracted so that part of the inner tube 102 is now within the handle 106, as shown in FIG. 5a. In this position, the sleeve has also been positioned over and around the infants head so that the inserter 100 may be removed.

When the inserter 100 has been removed, as shown in FIGS. 6a and 6b, the inner tube 102 is returned to its final unretracted position away from the handle 106. In this position, the moveable collar 132 remains within the handle 106 without extending past the distal ends of the flat springs 138, while the distal ends of the flat springs 138 are now in contact with the central rod 122 instead of the inner tube 102, since the inner tube 102 is no longer retracted towards the handle 106. The flat springs 138 engaging the central rod 122 thus form a physical barrier or obstruction that prevent the inner tube 102 from sliding proximally over the central rod 122, thereby preventing the inner tube 102 from being able to be retracted towards the handle 106 a second time. In this way, reuse of the device 10 is no longer possible.

In another embodiment, the device 10 is provided with tactile and auditory feedback to inform a user when the inner tube 102 has reached the fully retracted position. Such feedback is given by providing a button 134 on a moveable part in the handle 106 of the device 10, the moveable part being configured to be moved by the inner tube 102 as the inner tube 102 retracts, such that the button 134 becomes aligned with and pops up at an opening 116 provided on the handle 106 when the inner tube 102 is fully retracted. In the first embodiment of the device 10, as shown in FIGS. 1 to 6b, the moveable part that the button 134 is provided on is the moveable collar 132 described above. The button 134 is attached to the moveable collar 132 via a button biaser 135 that biases the button 134 outwardly away from the moveable collar 132. The button biaser 135 in the first embodiment is a flat spring.

In the first embodiment, the button 134 is positioned on the moveable collar 132 at a location such that before use of the device 10, as shown in FIGS. 1 to 3b, the button 134 is located within the handle 106 and hidden from view. The button biaser 135 biases the button 134 against an inner wall of the handle 106, away from the moveable collar 132. As described above, at this position, the moveable collar 132 is positioned relative to the stationary collar 136 such that the distal end of the moveable collar 132 extends beyond the distal end of the stationary collar 136, and the inner tube 102 is at an initial unretracted position. During use of the device 10, when the moveable collar 132 is pushed proximally into the handle 106 by the retracting inner tube 102 as shown in FIGS. 4a to 4c, the button 134 is similarly pushed proximally into the handle 106. The button 134 continues to move within the handle 106, being biased by the button biaser 135 against the inner wall of the handle 106 until just before the inner tube 102 has reached the fully retracted position as shown in FIGS. 5a and 5b.

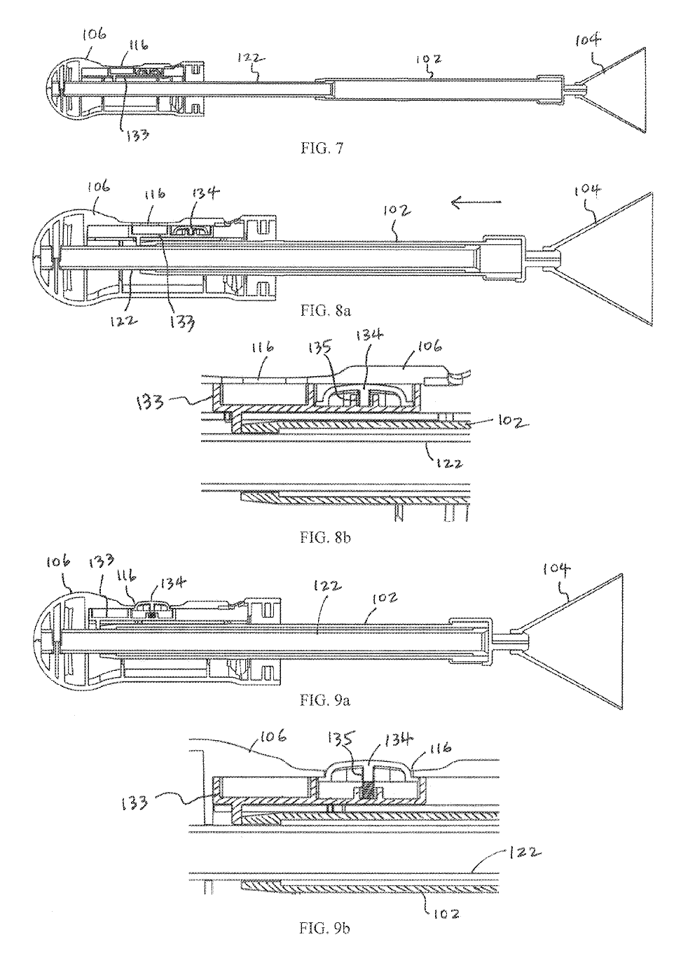

In a second embodiment of the device 10, as shown in FIGS. 7 to 9b, the moveable part that the button is provided on is a moveable bar 133 provided in the handle 106, between an inner wall of the handle 106 and the central rod 122. The button 134 is attached to the moveable bar 133 via a button biaser 135 that biases the button 134 outwardly away from the moveable bar 133.

In the second embodiment, before use of the device 10, the button 134 is located within the handle 106 and hidden from view. During use of the device 10, as shown in FIGS. 8a and 8b, the moveable bar 133 is pushed proximally into the handle 106 by the retracting inner tube 102, thereby moving the button 134 proximally into the handle 106. The button 134 continues to move within the handle 106, being biased by the button biaser 135 against the inner wall of the handle 106 until just before the inner tube 102 has reached the fully retracted position as shown in FIGS. 9a and 9b.

For all embodiments of the button 134, the opening 116 in the handle 106 is located on the handle 106 such that when the inner tube 102 is fully retracted, the button 134 becomes aligned with the opening 116. In this position, the button 134 pops up into the opening 116 as a result of the button biaser 135 biasing the button 134 outwardly. The button 134 is prevented from falling out of the opening 116 by configuring the button 134 to have a base that is larger than the size of the opening 116. The part of the button 134 that pops up at the opening 116 is necessarily smaller than the size of the opening in order for it to be able to fit through the opening 116.

When the button 134 pops up into the opening 116, tactile and auditory feedback comprising touch and sound are given to the user, thereby alerting to the user to the fact that the device 10 has reached a sufficient depth and no further pushing on the handle 106 of the inserter 100 is required. The advantage of providing tactile and auditory feedback is that such feedback does not require the user to actively look for an indication that the device 10 has reached a sufficient depth, which the user may inadvertently omit to do, and therefore inadvertently continue to exert unnecessary force on the handle 106.

Besides providing tactile and auditory feedback when the button 134 pops up in the opening 116, in the first embodiment, the button 134 in the opening 116 also provides a mechanical lock that prevents the moveable collar 132 from being moved distally to engage the flat springs 138 and allow retraction of the inner tube 102 again. The button 134 in the opening 134 thereby provides another safeguard to prevent the device 10 from being reset for a second use.

Whilst there has been described in the foregoing description exemplary embodiments of the present invention, it will be understood by those skilled in the technology concerned that many variations in details of design, construction and/or operation may be made without departing from the present invention. For example, instead of the flat springs 138, the elastic projection may comprise one or more cantilever arms integrally formed with the stationary collar 136. The elastic projection 138 may also be integrally formed with the handle 106 or directly attached to the handle 106 instead of via the stationary collar 136. Instead of using a flat spring or a compression spring as the button biaser 135, the button biaser 135 may be integrally formed with the button 134 and the moveable part as a elastically bendable cantilever arm attached to the moveable part and suspending the button 134 at its free end such that when the inner tube 102 has been fully retracted, the button 134 moves into the opening 116 in a snap-fit manner as a result of the cantilever arm elastically returning to its original shape. The device may also be configured to provide the moveable part as a moveable bar 133 in addition to the moveable collar 132.

1. A device for assisting delivery of an infant during childbirth, the device comprising:
a sleeve configured to be positioned over and around the infant's head for exerting traction on the infant therewith; and
an inserter comprising
a handle provided at a proximal end of the inserter,
flexible arms attached to the handle and configured to engage and position the sleeve over and around the infant's head,
a central rod attached to the handle,
an inner tube provided around the central rod at a distal end of the inserter such that the inner tube is retracted over the central rod when a force is applied to a soft cone provided at a distal end of the inner tube, the soft cone being configured to be placed on the infant's head to position the device for inserting the sleeve over and around the infant's head, and
a reuse prevention feature configured to prevent the inner tube from being retracted a second time after a single use of the device.

2. The device of claim 1, wherein the reuse prevention feature comprises
a moveable collar provided at least partially within the distal end of the handle around the central rod, the moveable collar configured to be moved by the inner tube, and
an elastic projection attached to the handle, the elastic projection extending distally and configured to engage the central rod,
wherein, before use of the device, the moveable collar engages the elastic projection, and after a single use of the device, the moveable collar is moved by the inner tube away from the elastic projection such that the elastic projection engages the central rod when the inner tube is returned to an unretracted position to prevent a second retraction of the inner tube.

3. The device of claim 2, wherein the elastic projection comprises a number of flat springs projecting from a distal end of a stationary collar attached to a distal end of the handle around the moveable collar, the distal end of each flat spring being biased inwardly towards the central rod.

4. The device of claim 1, further comprising an opening provided in the handle and a button attached to and biased outwardly from a moveable part provided in the handle, the moveable part configured to be moved by the inner tube when the inner tube is retracted during use of the device such that the button is aligned with and pops up in the opening when the inner tube is fully retracted.

5. The device of claim 4, wherein the moveable part is the moveable collar.

6. The device of claim 4, further comprising a button biaser configured to attach and bias the button outwardly away from the moveable part.
